(19) United States
(12) Patent Application Publication
(10) Pub. No.: US 2020/0046414 A1
(43) Pub. Date: Feb. 13, 2020

(54) SYSTEMS, METHODS, AND COMPUTER-READABLE MEDIA FOR CONTROLLING ABLATION ENERGY DELIVERY

(71) Applicant: Covidien LP, Mansfield, MA (US)

(72) Inventor: Joseph D. BRANNAN, Lyons, CO (US)

(21) Appl. No.: 16/534,025

(22) Filed: Aug. 7, 2019

Related U.S. Application Data
(60) Provisional application No. 62/717,038, filed on Aug. 10, 2018.

(51) Int. Cl. A61B 18/00; A61B 18/18; A61B 34/00

(52) U.S. Cl. CPC A61B 18/00; A61B 18/1815; A61B 34/25; A61B 2018/00702; A61B 2018/00577; A61B 2018/00511; A61B 2018/00708; A61B 2018/00642; A61B 2018/00541; A61B 2018/00529; A61B 2018/00761

(57) ABSTRACT

A method for controlling microwave ablation energy delivery includes receiving a setting entered via a user interface. An energy delivery profile is generated based on the setting, with the energy delivery profile defining an energy delivery amount adjustment to be made based at least in part on an elapsing of an amount of time relative to a reference point. Energy is delivered according to the energy delivery profile, with an amount of energy being delivered being adjusted based on the energy delivery amount adjustment when the amount of time relative to the reference point has elapsed.

### CROSS-REFERENCE TO RELATED APPLICATIONS

This application claims the benefit of the filing date of provisional U.S. Patent Application No. 62/717,038 filed, Aug. 10, 2018.

### INTRODUCTION

The present disclosure relates to tissue ablation. More specifically, the present disclosure describes systems, methods, and computer-readable media for controlling ablation energy delivery.

### BACKGROUND

Heating tissue with thermally ablative tools can cause abrupt phase transition of physiological water from liquid to gas. These phase transitions can cause small to large cavitation within the tissue surrounding the ablative tool. Large cavitations can have a deleterious effect upon procedural outcomes. For instance, large cavitations can cause distortion in the energy field and thus in the ablation zone shape or size, which can reduce the efficacy of the procedure. Large cavitations can also cause transport of diseased tissue outside of a heating or ablative profile or rupture of vascular structures, which can cause bleeding. In view of the foregoing, a need exists for improved systems and methods for controlling ablation energy delivery.

### SUMMARY

In one aspect, this disclosure describes a method for controlling microwave ablation energy delivery. The method includes receiving a setting entered via a user interface. An energy delivery profile is generated based on the setting, with the energy delivery profile defining an energy delivery amount adjustment to be made based at least in part on an elapsing of an amount of time relative to a reference point. Energy is delivered according to the energy delivery profile, with an amount of energy being delivered being adjusted based on the energy delivery amount adjustment when the amount of time relative to the reference point has elapsed.

In some embodiments, the setting includes an ablation zone size setting.

In further embodiments, the setting includes a tissue type setting.

In other embodiments, the setting includes an aggressiveness setting.

In still other embodiments, the generating the energy delivery profile includes setting a step time based on the aggressiveness setting.

In some embodiments, the generating the energy delivery profile includes setting a step magnitude based on the aggressiveness setting.

In further embodiments, the step magnitude is set so as to have an inversely proportional relationship with the aggressiveness setting.

In other embodiments, the setting includes an ablation speed setting.

In still other embodiments, the generating of the energy delivery profile includes (1) defining a plurality of steps of the energy delivery profile; (2) defining a plurality of step times corresponding to the plurality of steps, respectively; and (3) defining a plurality of energy delivery amounts corresponding to the plurality of steps, respectively.

In some embodiments, the amount of energy being delivered is adjusted based on the plurality of step times and the plurality of energy delivery amounts.

In further embodiments, the setting includes an ablation zone setting and the method further comprises ceasing the delivering of the energy based on the ablation zone size setting, before each of the plurality of step times elapses.

In other embodiments, the setting includes an ablation zone setting and the generating of the energy delivery profile includes determining, based on the ablation zone size setting, a final step time among the plurality of steps of the energy delivery profile.

In still other embodiments, the generating the energy delivery profile defines the energy delivery amount adjustment to be made further based at least in part on feedback from a sensor, and the amount of energy being delivered is adjusted based on the energy delivery amount adjustment based at least in part on the feedback from the sensor.

In some embodiments, the method further comprises determining, based on at least one of sensor feedback or the amount of time elapsed relative to the reference point, when to cease energy delivery for at least a predetermined amount of time; and ceasing energy delivery based on a result of the determining.

In further embodiments, the setting includes an ablation zone size setting and the generating of the energy delivery profile includes defining a plurality of steps of the energy delivery profile based on the ablation zone size setting.

In other embodiments, the generating of the energy delivery profile includes defining a plurality of steps of the energy delivery profile based on a maximum amount of energy a generator is capable of delivering.

In still other embodiments, the generating of the energy delivery profile includes defining a plurality of steps of the energy delivery profile based on a step time.

In some embodiments, the generating of the energy delivery profile includes defining a plurality of energy delivery amounts based on a step time.

In further embodiments, the setting includes a tissue type setting and the generating of the energy delivery profile includes defining a step time based on the tissue type setting.

In other embodiments, the tissue type setting is received by way of a tissue type control of the user interface by which the tissue type setting can be set to a tissue type among a plurality of tissue types including at least a lung, a liver, and a kidney.

### BRIEF DESCRIPTION OF THE DRAWINGS

Various aspects and features of the present microwave ablation system and methods are described herein below with references to the drawings, wherein:

FIG. 1 is a schematic diagram of a microwave ablation system, according to an embodiment of the present disclosure;

FIG. 2 is a schematic diagram of a computing device which forms part of the microwave ablation system of FIG. 1 in accordance with some embodiments;

FIG. 3 is a flowchart illustrating an example procedure for using the microwave ablation system of FIG. 1 to perform microwave ablation;

FIG. 4 is a flowchart illustrating an example procedure for using the microwave ablation system of FIG. 1 to generate an energy delivery profile;

FIG. 5 shows an example energy delivery profile that may be generated according to the procedure of FIG. 4; and

FIG. 6 is a flowchart illustrating an example procedure for using the microwave ablation system of FIG. 1 to deliver energy according to an energy delivery profile generated according to the procedure of FIG. 4.

### DETAILED DESCRIPTION

The present disclosure is directed to systems, methods, and computer-readable media for controlling ablation energy delivery. In one aspect, the systems, methods, and computer-readable media of the present disclosure provide an energy delivery algorithm and user interface that maximize ablative zone predictability, simplify user configuration of an ablation system, and minimize abrupt phase transition of physiological fluids during energy delivery. According to various embodiments, the systems, methods, and computer-readable media of the present disclosure facilitate the control of, and/or reduction in, the rate of change of tissue temperature with respect to time, for instance, during an initial portion of an ablation cycle. Controlling or lowering the rate of change of tissue temperature with respect to time allows fluid to move peripherally away from the ablative tool prior to undergoing phase change. By “pushing” water away, or “drying out” the tissue in proximity to the ablative tool prior to reaching phase change temperature or pressure, the energy algorithm provided herein reduces the abrupt phase transition of physiological water from liquid to gas and thus reduces the likelihood and intensity of cavitation. The energy algorithm provided herein thus minimizes the following deleterious effects that can otherwise be caused by cavitations: (1) distortion in the energy field and in the ablation zone shape or size, (2) the transport of diseased tissue outside of a heating or ablative profile, and (3) the rupture of vascular structures.

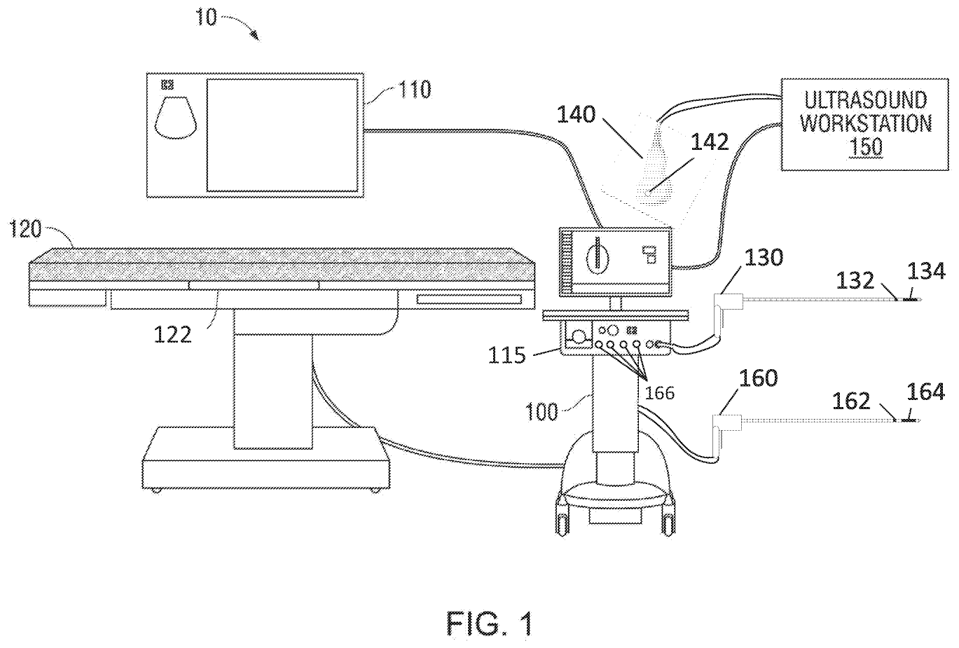

Referring now to FIG. 1, the present disclosure is generally directed to a treatment system 10, which includes a computing device 100, a display 110, a table 120, an ablation probe 130, an ultrasound sensor 140, an ultrasound workstation 150, and a remote temperature probe (RTP) 160. The computing device 100 may be, for example, a laptop computer, a desktop computer, a tablet computer, and/or another similar device. The computing device 100 may be configured to control and/or receive data from an electrosurgical generator 115, a peristaltic pump (not expressly shown in FIG. 1), a power supply (not expressly shown in FIG. 1), and/or any other accessories and peripheral devices relating to, or forming part of, the system 10. The computing device 100 further controls and/or receives data from the ultrasound workstation 150 and the RTP 160.

A front panel of the generator 115 provides a user interface having one or more input controls 166 (for example, a rotary knob, a button, a switch, a touch panel, or any other type of input control) by which one or more respective settings can be entered. Example types of input controls 166 and respective settings include, without limitation, an ablation zone size setting, a tissue type setting, an aggressiveness setting, and/or an ablation speed setting. In various embodiments, and as described in further detail elsewhere herein, the input controls 166 can be used to adjust various settings that control generation of ablation energy by the generator 115, and delivery of ablation energy from the generator 115 to patient tissue by way of the radiating portion 134 of the ablation probe 130. The input controls 166 thus facilitate control of ablation energy generation and delivery in a manner that maximizes ablative zone predictability and minimizes abrupt phase transition of physiological fluids during energy delivery. Additionally, although the input controls 166 are shown in FIG. 1 as being located on the front panel of the generator 115, in other embodiments, any one or more of the input controls 166 may be located in another part of the system 10, such as the computing device 100, the display 110, and/or the ultrasound workstation 150.

In some embodiments, the generator 115 includes input controls 166 corresponding to a subset (fewer than all) of the following settings: an ablation zone size setting, a tissue type setting, an aggressiveness setting, and/or an ablation speed setting, and the generator 115 includes logic that controls the generation of an energy delivery profile (described in further detail below) based on the value(s) of the available setting(s). For instance, in an embodiment where the only input control 166 that the generator 115 includes is the input control 166 for the ablation zone size setting, the generator 115 might set a common step time for each of the steps of the energy delivery profile and might set a common energy delivery magnitude increase (e.g., the relative increase in energy delivery magnitudes from one step to the following step) for each of the steps. In this case, the generator 115 might be configured to progress further into the energy delivery profile for progressively larger ablation zone sizes. As another example, in an embodiment where the generator 115 includes an input control 166 for the ablation zone size setting and another input control 166 for the aggressiveness setting (or an input control 166 for the ablation speed setting), the generator 115 might be configured to shorten the step time of the steps and/or increase the energy delivery magnitudes of the steps for a more aggressive setting (or a faster ablation speed setting).

The display 110 is configured to output instructions, images, and messages relating to the microwave ablation procedure. The computing device 100 may also include a display that may be configured to output instructions, images, and/or messages relating to the microwave ablation procedure. The table 120 may be, for example, an operating table or other table suitable for use during a surgical procedure. The table 120 includes an electromagnetic (EM) field generator 122 that is used to generate an EM field during the microwave ablation procedure. The EM field generator 122 forms part of an EM tracking system used to track the positions of instruments, such as the ablation probe 130, the ultrasound sensor 140, and/or the RTP 160, within the EM field. The EM field generator 122 may include various components, such as a specially designed pad to be placed under, or integrated into, an operating table or patient bed. An example of such an EM tracking system is the AURORA system sold by Northern Digital Inc. The EM tracking system further includes various EM sensors 132, 142, 162 coupled to or included in instruments, as described further below. The EM tracking system provides data regarding the EM field and the tracked positions of the EM sensors 132, 142, 162 to the computing device 100. The computing device 100 uses the data received from the EM tracking system to determine positions of the instruments relative to each other and to marked objects, as further described below.

The ablation probe 130 is a surgical instrument having a microwave ablation antenna which is used to ablate tissue. The ablation probe 130 receives microwave energy from the generator 115. The ablation probe 130 includes an EM sensor 132 by means of which the EM tracking system tracks the position of the ablation probe 130. An example method of tracking the location of the ablation probe 130 includes using the EM tracking system, which tracks the location of the ablation probe 130 by tracking the EM sensor 132 coupled to or incorporated within the ablation probe 130. Various types of sensors may be used, such as a printed sensor, the construction and use of which is more fully described in U.S. Patent Appl. Publ. No. 2016/0174873, entitled MEDICAL INSTRUMENT WITH SENSOR FOR USE IN A SYSTEM AND METHOD FOR ELECTROMAGNETIC NAVIGATION, filed Oct. 22, 2015, by Greenburg et al. the entire contents of which is incorporated herein by reference. The ablation probe 130 further includes a radiating portion 134 from which microwave energy is emitted when the ablation probe 130 is activated. The ablation probe 130 is used to ablate a lesion or tumor (hereinafter referred to as a “target”) by using electromagnetic radiation or microwave energy to heat tissue in order to denature or kill cancerous cells. The construction and use of a system including such an ablation probe 130 are more fully described in U.S. Patent Appl. Publ. No. 2016/0058507, entitled MICROWAVE ABLATION SYSTEM, filed on Aug. 18, 2015, by William J. Dickhans, U.S. Pat. No. 9,247,992, entitled MICROWAVE ABLATION CATHETER AND METHOD OF UTILIZING THE SAME, filed on Mar. 15, 2013, by Ladtkow et al., and U.S. Pat. No. 9,119,650, entitled MICROWAVE ENERGY-DELIVERY DEVICE AND SYSTEM, filed on Mar. 15, 2013, by Brannan et al., the contents of each of which are hereby incorporated by reference in its entirety.

The ultrasound sensor 140 may be any ultrasound device which, in conjunction with the ultrasound workstation 150, provides ultrasound data and/or images to the computing device 100. In embodiments, the ultrasound sensor 140, such as an ultrasound wand or transducer, may be used to image the patient's body during the microwave ablation procedure to visualize the location of the surgical instruments, such as the ablation probe 130 and/or the RTP 160, and/or structures or objects inside the patient's body. The ultrasound sensor 140 has an EM tracking sensor 142 included therein or coupled thereto, for example, a clip-on sensor or a sticker sensor. As described further below, the ultrasound sensor 140 may be positioned in relation to the ablation probe 130 and/or the RTP 160 such that the ablation probe 130 or the RTP 160 is at an angle to the ultrasound image plane, thereby enabling the clinician to visualize the spatial relationship of the ablation probe 130 and/or the RTP 160 with the ultrasound image plane and with the objects being imaged. The EM tracking system may also track the position of the ultrasound sensor 140 within the EM field to enable the computing device 100 to determine a location of the ultrasound image plane relative to marked objects within the EM field, as described further below. In some embodiments, one or more ultrasound sensors 140 may be placed inside the body of the patient. The EM tracking system may then track the location of such ultrasound sensors 140, the ablation probe 130, and/or the RTP 160 inside the body of the patient.

The RTP 160 may be any surgical device that includes an EM sensor 162 and a temperature sensor 164. For the purpose of clarity, in the embodiments described below, RTP 160 is described as a surgical instrument dedicated to the purpose of monitoring temperature. However, those skilled in the art will appreciate that RTP 160 may form part of another surgical instrument, such as a second ablation probe, a vessel sealing device, a surgical stapler, etc., and is used to monitor temperature during the microwave ablation procedure prior to or after performing another function.

While the present disclosure describes the use of system 10 in a surgical environment, it is also envisioned that some or all of the components of system 10 may be used in alternative settings, for example, an imaging laboratory and/or an office setting. Also, while the system 10 shown in FIG. 1 and described herein is generally usable for percutaneous procedures, it is also envisioned that other types of microwave ablation systems, for example, an endobronchial microwave ablation system and/or the like (which may include some or all of the components of system 10), may be used as an alternative to the system 10. Additionally, those skilled in the art will appreciate that various other surgical instruments or tools, such as vessel sealing devices, surgical staplers, etc., may also be equipped with an EM sensor and used during the performance of a microwave ablation treatment procedure.

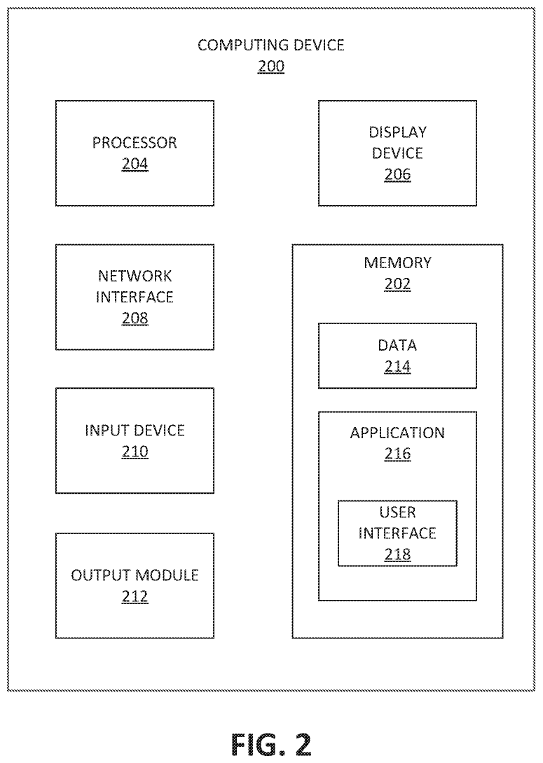

FIG. 2 is a schematic block diagram of a computing device 200 that may be employed in accordance with various embodiments described herein. Although not explicitly shown in FIG. 1, in some embodiments, the computing device 200, or one or more of the components thereof, may further represent one or more components (e.g., the computing device 100, the electrosurgical generator 115, the ultrasound workstation 150, and/or the like) of the system 10. The computing device 200 may, in various embodiments, include one or more memories 202, processors 204, display devices 206, network interfaces 208, input devices 210, and/or output modules 212. The memory 202 includes non-transitory computer-readable storage media for storing data and/or software that is executable by the processor 204 and which controls the operation of the computing device 200. In embodiments, the memory 202 may include one or more solid-state storage devices such as flash memory chips. Alternatively, or in addition to the one or more solid-state storage devices, the memory 202 may include one or more mass storage devices connected to the processor 204 through a mass storage controller (not shown in FIG. 2) and a communications bus (not shown in FIG. 2). Although the description of computer readable media included herein refers to a solid-state storage, it should be appreciated by those skilled in the art that computer-readable storage media may be any available media that can be accessed by the processor 204. That is, computer readable storage media includes non-transitory, volatile and non-volatile, removable and non-removable media implemented in any method or technology for storage of information such as computer-readable instructions, data structures, program modules or other data. Examples of computer-readable storage media include RAM, ROM, EPROM, EEPROM, flash memory or other solid state memory technology, CD-ROM, DVD, Blu-Ray or other optical storage, magnetic cassettes, magnetic tape, magnetic disk storage or other magnetic storage devices, or any other medium which may be used to store the desired information and which can be accessed by computing device 200.

In some embodiments, the memory 202 stores data 214 and/or an application 216. In some aspects the application 216 includes a user interface component 218 that, when executed by the processor 204, causes the display device 206 to present a user interface, for example a graphical user interface (GUI) (not shown in FIG. 2). The network interface 208, in some embodiments, is configured to couple the computing device 200 and/or individual components thereof to a network, such as a wired network, a wireless network, a local area network (LAN), a wide area network (WAN), a cellular network, a Bluetooth network, the Internet, and/or another type of network. The input device 210 may be any device by means of which a user may interact with the computing device 200. Examples of the input device 210 include without limitation a mouse, a keyboard, a touch screen, a voice interface, a computer vision interface, and/or the like. The output module 212 may, in various embodiments, include any connectivity port or bus, such as, for example, a parallel port, a serial port, a universal serial bus (USB), or any other similar connectivity port known to those skilled in the art.

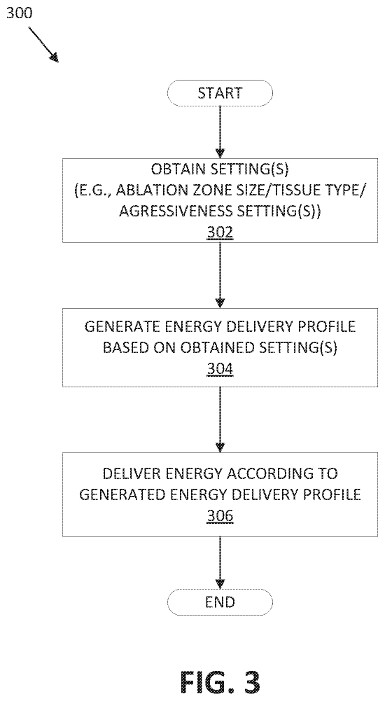

FIG. 3 is a flowchart illustrating an example procedure 300 for using the microwave ablation system 10 of FIG. 1 to perform microwave ablation. At block 302, one or more settings are received via the user interface of the front panel of the generator 115 or, more particularly, via the one or more input controls 166. The input controls 166 are used to adjust various settings that control generation of ablation energy by the generator 115, and delivery of ablation energy from the generator 115 to patient tissue by way of the radiating portion 134 of the ablation probe 130, in a manner that maximizes ablative zone predictability and minimizes abrupt phase transition of physiological fluids during energy delivery. Example types of input controls 166 and respective settings include, without limitation, an ablation zone size setting, a tissue type setting, an aggressiveness setting, and/or an ablation speed setting, with each such setting having a particular impact on one or more aspects of the energy delivery profile, such as the number of steps, the respective energy delivery magnitudes of the steps, the step times for the steps, and/or the like, as described in further detail below. For example, the ablation zone size setting may have a range of possible values including 1 millimeter to 5 millimeters in 0.5 millimeter increments; the tissue type setting may have a range of possible values including lung, liver, and kidney; the aggressiveness setting may have a range of possible values including cautious, balanced, and aggressive; the ablation speed setting may have a range of possible values including slow, moderate, and fast. In some examples, the cautious, balanced, and aggressive settings may correspond to the slow, moderate, and fast settings, respectively.

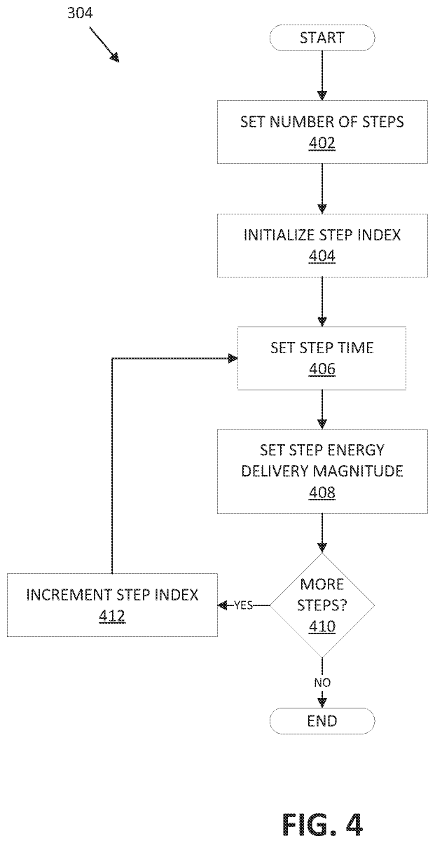

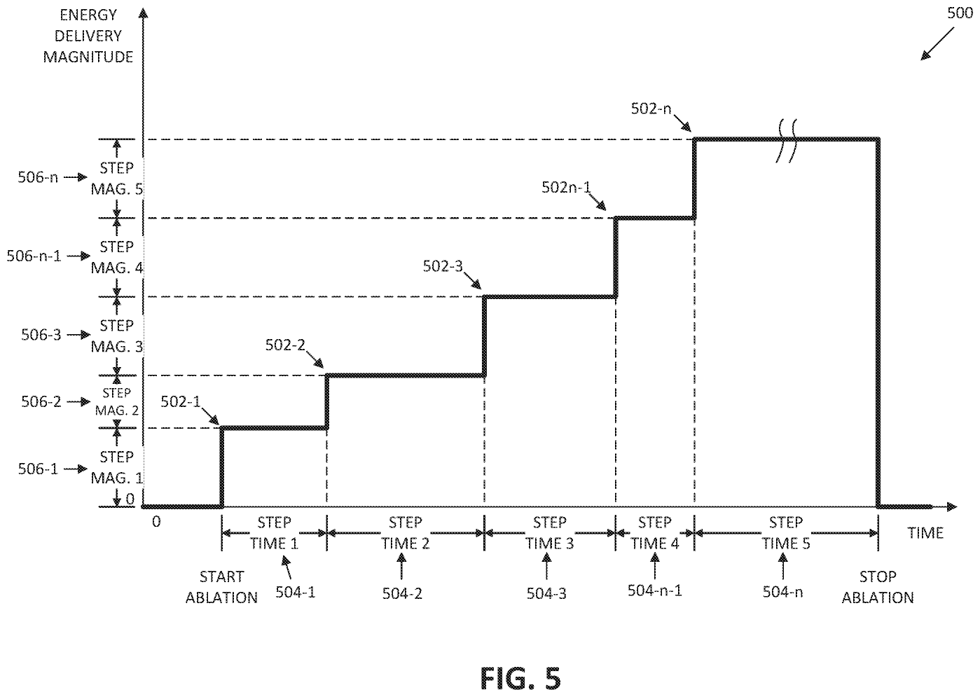

At block 304, the generator 115 generates an energy delivery profile based on the one or more settings received at block 302. The energy delivery profile defines one or more energy delivery amount adjustments to be made (or energy delivery magnitudes at which energy is to be delivered) based at least in part on an elapsing of an amount of time relative to a reference point, feedback from one or more sensors, and/or other types of criteria. For example, the energy delivery profile may define energy delivery amount adjustments (which may be referred to as steps) to be made at various times (which may be referred to as step times) relative to the reference point, to effectively taper the energy delivery over time. The reference point, in various embodiments, may be based on an event. For example, the reference point may be based on initiation of energy delivery, in which case the reference point can be a time at which energy delivery is initiated by the generator 115. The reference point may alternatively be a time at which the energy delivery amount was most recently adjusted, and/or any other type of reference point. The energy delivery profile, in some embodiments, may also define energy delivery amount adjustments to be made based at least in part on feedback provided to the generator 115 from one or more sensors, such as tissue temperature sensors, reflected energy sensors, tissue pressure sensors, and/or other types of sensors that sense one or more aspects corresponding to the progress of an ablation procedure. In some examples, for instance, the energy delivery profile may be scaled in energy and/or time based on feedback from the one or more sensors. Further details regarding the generating of the energy delivery profile at block 304 are shown in FIG. 4 and FIG. 5 and described below.

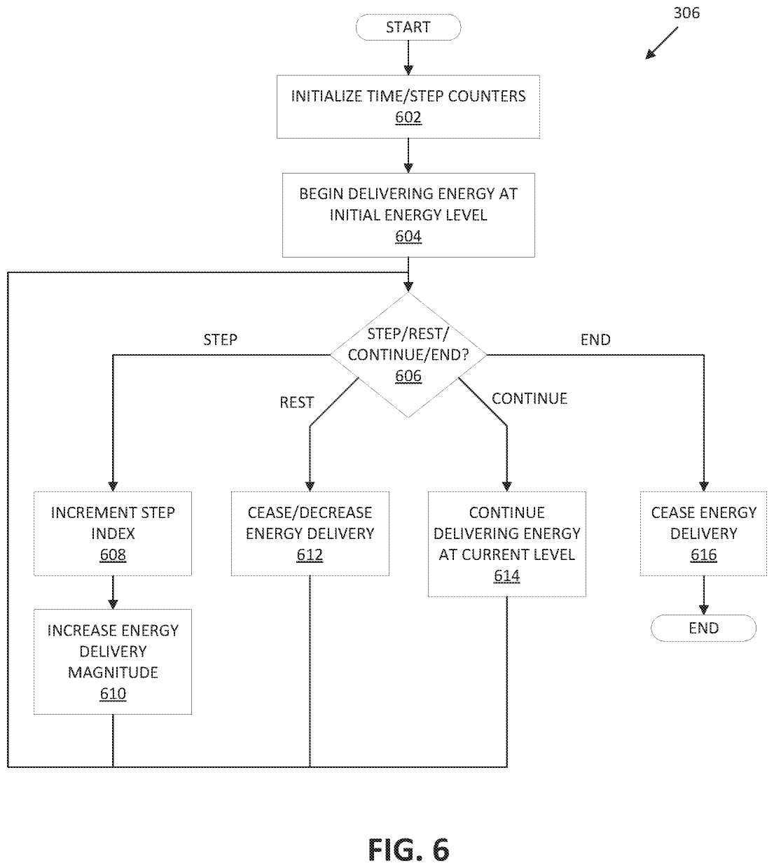

At block 306, the generator 115 delivers energy to patient tissue according to the energy delivery profile, by way of the radiating portion 134 of the ablation probe 130. In an embodiment where the energy delivery profile is at least partially based on the elapsing of time, the amount of energy being delivered at block 306 is adjusted based on the energy delivery amount adjustment, which is defined in the energy delivery profile, when the amount of time relative to the reference point, which is also defined in the energy delivery profile, has elapsed. Further details regarding the delivery of energy block 306 are shown in FIG. 6 and described below.

With reference to FIG. 4 and FIG. 5, an example procedure 304 for using the microwave ablation system 10 of FIG. 1 to generate an energy delivery profile 500 will be described. As described above in the context of block 304 of FIG. 3, in various embodiments, the generator 115 generates the energy delivery profile based on the one or more settings received at block 302. To that end, at block 402, the generator 115 sets a number of steps based on the setting(s) received at block 302. For instance, as shown in the example energy delivery profile 500 of FIG. 5, which may be generated according to the procedure 304 of FIG. 4, the generator 115 may set a number of steps as n, in which case the energy delivery profile 500 defines n steps 502-1, 502-2, . . . , 502-n (collectively, steps 502). For example, the generator 115 may set the number of steps n based on the ablation size setting (with the number of steps n having a proportional relationship with the value of the ablation zone size setting), the maximum amount of energy deliverable by the generator 115 (with the number of steps n having a proportional relationship with the maximum amount of energy deliverable by the generator 115), the step time set at block 406, which is described below (with the number of steps n having a proportional relationship with the step time), and/or the like. In some examples, the generator 115 may, in generating an energy delivery profile, determine, based on the ablation zone size setting, a final step time for the energy delivery profile, such as by elongating the final step time to achieve the desired ablation zone size.

At block 404, the generator 115 initializes a step index s by setting s equal to 1 to correspond to the first step 502-1 of the n steps 502. At block 406, based on the setting(s) received at block 302, the generator 115 sets a first step time 504-1 corresponding to the first step 502-1, as shown in the example energy delivery profile 500 of FIG. 5. For example, the generator 115 may set the step time 504-1 based on the tissue type setting, a setting indicating a proximity of the target tissue to a critical structure of the patient, the aggressiveness setting (with the step time 504-1 having an inversely proportional relationship with the aggressiveness setting), and/or the like.

Setting the step time based at least in part on the aggressiveness setting, in some examples, may also impact the number of steps of the energy delivery profile. For instance, for an aggressiveness setting set to aggressive, the generator 115 might generate an energy delivery profile having three steps—a first step having a step time of 45 seconds and an energy delivery magnitude of 45 watts, a second step having a step size of 30 seconds and an energy delivery magnitude of 75 watts, and a third step having a step size of 1 minute and an energy delivery magnitude of 100 watts. For an aggressiveness setting set to cautious, however, the generator 115 might generate an energy delivery profile having only two steps—a first step having a step time of 2 minutes and an energy delivery magnitude of 45 watts, and a second step having a step size of 2 minutes and an energy delivery magnitude of 75 watts.

One example of how the step time might be determined based at least in part on the tissue type setting is as follows. In the following example, the ablation zone size setting is set to 3 centimeters. For a tissue type setting set to liver tissue, the generator 115 might generate an energy delivery profile having three steps—a first step having a step time of 1 minute and an energy delivery magnitude of 45 watts, a second step having a step size of 2 minutes and an energy delivery magnitude of 75 watts, and a third step having a step size of 30 seconds and an energy delivery magnitude of 100 watts. For a tissue type setting set to lung tissue, the generator 115 might generate an energy delivery profile having two steps—a first step having a step time of 2 minutes and an energy delivery magnitude of 75 watts, and a second step having a step size of 30 seconds and an energy delivery magnitude of 100 watts. For a tissue type setting set to kidney tissue, the generator 115 might generate an energy delivery profile having three steps—a first step having a step time of 1 minute and an energy delivery magnitude of 45 watts, a second step having a step size of 1 minute and an energy delivery magnitude of 75 watts, and a third step having a step size of 1 minute and an energy delivery magnitude of 100 watts.

At block 408, based on the setting(s) received at block 302, the generator 115 sets a first energy delivery magnitude 506-1 corresponding to the first step 502-1 and the first step time 504-1. In general, the first energy delivery magnitude 506-1 indicates a magnitude at which the generator 115 is to delivery energy during the first step time 504-1. For example, the generator 115 may set the energy delivery magnitude 506-1 based on the step time 504-1 set at block 404 (with the energy delivery magnitude 506-1 having a proportional relationship with the step time 504-1), and/or the like.

At block 410, the generator 115 determines whether an additional step time and an additional energy delivery magnitude are to be set for a further step of the n steps 502 of the energy delivery profile. In one example, the generator 115 makes this determination by comparing the current value of the step index s to the number of steps n that was set at block 402. In this case, if the step index s is less than the number of steps n, then the generator 115 determines that an additional step time and an additional energy delivery magnitude are to be set for a further step of the n steps 502 of the energy delivery profile. If the step index s is equal to the number of steps n, then the generator 115 determines that no additional step time or additional energy delivery magnitude are to be set for any further step of the n steps 502 of the energy delivery profile.

If the generator 115 determines that an additional step time and an additional energy delivery magnitude is to be set (“YES” at block 410), then control passes to block 412, where the step index is incremented by 1 to correspond to the next step among the steps 502 of the energy delivery profile. Control then passes back through block 406 and block 408 to set the additional step time and additional energy delivery magnitude in the manner described above. The generator 115 thus repeats the procedures of blocks 406 and 408 for each step of the n steps 502 of the energy delivery profile. Referring back to block 410, if the generator 115 determines that no additional step times and energy delivery magnitudes are to be set (“NO” at block 410), thereby indicating that generation of the energy delivery profile is complete, then the procedure 304 terminates and the generated energy delivery profile, such as the example profile 500 of FIG. 5, is ready for use to control energy delivery during an ablation procedure, for instance, as described in connection with FIG. 6. Although the example energy delivery profile 500 shown in FIG. 5 includes steps for which the energy delivery magnitude increases in a stepwise manner, this is provided by way of example and not limitation. In various embodiments, other types of energy delivery waveforms are also contemplated, such as a linearly tapered energy delivery profile, an energy delivery profile having exponentially increasing energy delivery magnitudes, and/or the like.

FIG. 6 is a flowchart illustrating an example procedure 306 for using the microwave ablation system 10 of FIG. 1 to deliver energy according to an energy delivery profile (e.g., the energy delivery profile 500 of FIG. 5) generated according to the procedure 304 of FIG. 4. At block 602, a time counter, t, and a step counter, s, are initialized, for instance, by setting the time counter t equal to zero and setting the step counter s equal to 1 to correspond to the first step of the n steps of the energy delivery profile that was generated according to the procedure 304 (FIG. 3 and FIG. 4). The time counter t, in some examples, is set to zero at block 602 and only begins to count upwards when energy delivery is initiated. In this manner, all time during the ablation procedure is measured relative to the initiation of energy delivery, and the time counter can be used as the temporal basis upon which to step through the various steps 502 of the energy delivery profile at predetermined step times 504.

At block 604, the generator 115 begins delivering energy at the initial energy magnitude, which is the first energy delivery magnitude 506-1 which corresponds to the first step 502-1 of the n steps 502 of the energy delivery profile. At block 606, the generator 115 determines whether to continue delivering energy at the current energy magnitude, or modify the energy delivery magnitude in some manner, such as by increasing the energy delivery magnitude, decreasing the energy delivery magnitude, or ceasing energy delivery. The generator 115 makes the determination at block 606 based at least in part upon the energy delivery profile that was generated according to the procedure 304 (FIG. 3 and FIG. 4). For instance, the generator 115 may make the determination at block 606 based solely on the current value of the time index t relative to the reference point at which the time index t was equal to zero. In this manner, the generator 115 may use the time index as the basis upon which to perform lookups into a table that defines when to advance through the steps 502 of the energy delivery profile 500. Alternatively, or in addition, the generator 115 may make the determination at block 606 based upon sensor feedback received from one or more sensors, as described above.

If the generator determines at block 606 to advance to the next step 502-2 in the energy delivery profile (“STEP” at block 606), then at block 608 the step index s is incremented by 1 to correspond to the next step (step 502-2 in this case) in the energy delivery profile 500. Then, at block 610, the generator 115 increases the energy delivery magnitude to the energy delivery magnitude that corresponds to step 502-2. Control then passes back to block 606. In this manner, the amount of energy being delivered may be adjusted based on the step times and energy delivery amounts corresponding to those step times.

Although not expressly shown in the energy delivery profile 500 of FIG. 5, the energy delivery profile may include one or more rest periods in between successive steps, for instance, to allow fluids time to move peripherally away from the ablation probe 130. In such a case, for example, the generator 115 might determine, based on sensor feedback and/or an amount of time that has elapsed relative to a reference point, when to cease energy delivery for at least a predetermined amount of time; and may cease energy delivery based on a result of that determining. If the generator determines at block 606 to begin a rest period (“REST” at block 606), then at block 612 the generator 115 decreases the energy delivery magnitude or ceases energy delivery to effectuate a rest period. Control then passes back to block 606.

If the generator 115 determines at block 606 that it should continue delivering energy at the current energy delivery magnitude (“CONTINUE” at block 606), then at block 614 the generator 115 continues delivering energy at the current energy delivery magnitude. Control then passes back to block 606.

If the generator 115 determines at block 606 that it should cease delivering energy (“END” at block 606), for instance indicating that the ablation procedure is completed, then at block 616 the generator 115 ceases energy delivery and the procedure 306 terminates. In some examples, the amount of progression into the energy delivery profile may depend upon the size of the ablation zone desired. For example, the step magnitudes may not vary, only the progression into or through the number of steps of the energy delivery waveform varies depending upon an ablation zone size setting selected by the user via the input control(s) 166. In other words, the generator 115 might determine how far to progress into the energy delivery profile based on the value of the ablation zone size setting, and may determine at block 606 to cease energy delivery before a full progression into the energy delivery profile, for instance at one or more steps prior to the final step of the energy delivery profile. For instance, the generator 115 may have generated an energy delivery profile that includes three steps—a first step having a step size of 1 minute and an energy delivery magnitude of 45 watts, a second step having a step size of 2 minutes and an energy delivery magnitude of 75 watts, and a third step having a step size of 30 seconds and an energy delivery magnitude of 100 watts. In this example, for an ablation zone size setting of 2 centimeters, the generator 115 might determine based on the value of the ablation zone size setting that it should only progress partially (e.g., 20 seconds) into the second step of the energy delivery profile and then should cease delivering energy. Alternatively, for the same energy delivery profile but for an ablation zone size setting of 3 centimeters, the generator 115 might determine that it should progress fully into the energy delivery profile and cease delivering energy after delivering 100 watts for 30 seconds. Similarly, the generator 115 might determine how far to progress into the energy delivery profile based on the value of the ablation zone size setting, and may determine at block 606 to continue delivering energy at the energy magnitude of the final step of the energy delivery profile (e.g., elongating the final step time) to achieve the desired ablation zone size. In the example noted above, for instance, the generator 115 might determine to deliver 100 watts for longer than 30 seconds.

The embodiments disclosed herein are examples of the disclosure and may be embodied in various forms. For instance, although certain embodiments herein are described as separate embodiments, each of the embodiments herein may be combined with one or more of the other embodiments herein. Specific structural and functional details disclosed herein are not to be interpreted as limiting, but as a basis for the claims and as a representative basis for teaching one skilled in the art to variously employ the present disclosure in virtually any appropriately detailed structure. Like reference numerals may refer to similar or identical elements throughout the description of the figures.

Throughout this description, the phrases “in an embodiment,” “in embodiments,” “in some embodiments,” or “in other embodiments” may each refer to one or more of the same or different embodiments in accordance with the present disclosure. A phrase in the form “A or B” means “(A), (B), or (A and B).” A phrase in the form “at least one of A, B, or C” means “(A); (B); (C); (A and B); (A and C); (B and C); or (A, B, and C).”

The foregoing description is only illustrative of the present microwave ablation systems and devices. Various alternatives and modifications can be devised by those skilled in the art without departing from the disclosure. Accordingly, the present disclosure is intended to embrace all such alternatives, modifications and variances. The embodiments described with reference to the attached drawing figures are presented only to demonstrate certain examples of the disclosure. Other elements, steps, methods, and techniques that are insubstantially different from those described above and/or in the appended claims are also intended to be within the scope of the disclosure.

What is claimed is:

1. A method for controlling ablation energy delivery, comprising:
receiving a setting entered via a user interface;
generating an energy delivery profile based on the setting, the energy delivery profile defining an energy delivery amount adjustment to be made based at least in part on an elapsing of an amount of time relative to a reference point; and
delivering energy according to the energy delivery profile, wherein an amount of energy being delivered is adjusted based on the energy delivery amount adjustment when the amount of time relative to the reference point has elapsed.

2. The method of claim 1, wherein the setting includes an ablation zone size setting.

3. The method of claim 1, wherein the setting includes a tissue type setting.

4. The method of claim 1, wherein the setting includes an aggressiveness setting.

5. The method of claim 4, wherein the generating the energy delivery profile includes setting a step time based on the aggressiveness setting.

6. The method of claim 4, wherein the generating the energy delivery profile includes setting a step magnitude based on the aggressiveness setting.

7. The method of claim 6, wherein the step magnitude is set so as to have an inversely proportional relationship with the aggressiveness setting.

8. The method of claim 4, wherein the setting includes an ablation speed setting.

9. The method of claim 1, wherein the generating of the energy delivery profile includes:
defining a plurality of steps of the energy delivery profile;
defining a plurality of step times corresponding to the plurality of steps, respectively; and
defining a plurality of energy delivery amounts corresponding to the plurality of steps, respectively.

10. The method of claim 9, wherein the amount of energy being delivered is adjusted based on the plurality of step times and the plurality of energy delivery amounts.

11. The method of claim 9, wherein the setting includes an ablation zone setting and the method further comprises:
ceasing the delivering of the energy, based on the ablation zone size setting, before each of the plurality of step times elapses.

12. The method of claim 9, wherein the setting includes an ablation zone setting and the generating of the energy delivery profile includes determining, based on the ablation zone size setting, a final step time among the plurality of steps of the energy delivery profile.

13. The method of claim 1,
wherein the generating the energy delivery profile defines the energy delivery amount adjustment to be made further based at least in part on feedback from a sensor, and
wherein the amount of energy being delivered is adjusted based on the energy delivery amount adjustment based at least in part on the feedback from the sensor.

14. The method of claim 1, further comprising:
determining, based on at least one of sensor feedback or the amount of time elapsed relative to the reference point, when to cease energy delivery for at least a predetermined amount of time; and
ceasing energy delivery based on a result of the determining.

15. The method of claim 1, wherein the setting includes an ablation zone size setting and the generating of the energy delivery profile includes defining a plurality of steps of the energy delivery profile based on the ablation zone size setting.

16. The method of claim 1, wherein the generating of the energy delivery profile includes defining a plurality of steps of the energy delivery profile based on a maximum amount of energy a generator is capable of delivering.

17. The method of claim 1, wherein the generating of the energy delivery profile includes defining a plurality of steps of the energy delivery profile based on a step time.

18. The method of claim 1, wherein the generating of the energy delivery profile includes defining a plurality of energy delivery amounts based on a step time.

19. The method of claim 1, wherein the setting includes a tissue type setting and the generating of the energy delivery profile includes defining a step time based on the tissue type setting.

20. The method of claim 19, wherein the tissue type setting is received by way of a tissue type control of the user interface by which the tissue type setting can be set to a tissue type among a plurality of tissue types including at least a lung, a liver, and a kidney.
